(19) United States
(12) Patent Application Publication
(10) Pub. No.: US 2007/0156228 A1
(43) Pub. Date: Jul. 5, 2007

(54) Prosthetic stent graft for treatment of abdominal aortic aneurysm

(76) Inventors: David C. Majercak, Stewartsville, NJ (US); Jin S. Park, Parsippany, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/324,682

(22) Filed: Jan. 3, 2006

(51) Int. Cl. A61F 2/06; A61F 2/94

(52) U.S. Cl. 623/1.13; 623/1.28; 623/1.31; 623/1.35

(57) ABSTRACT

A prosthetic stent graft including a trunk with a vascular graft bifurcated into two flow channels. The flow channels have a first taper angle, decreasing area along its length between the bifurcation point and an outlet. A leg section of the stent graft includes a tubular vascular graft. An inlet of the leg section has a deployed diameter at least about the same size or larger than a deployed diameter of the outlet of the flow channels. The leg section has its own taper angle, preferably less than or about equal to, and more preferably about equal to the taper angle of the flow channel. The deployed diameter of the leg section inlet is preferably about the same size or larger than the deployed diameter of the flow channels adjacent the bifurcation point of the trunk section, and the deployed diameter of flow channel outlets are preferably about the same size or smaller than the deployed diameter of the leg section at an equal distance from the leg section inlet as the outlet is from bifurcation point. Also provided according to the present invention is a method of in situ stent graft sizing for the treatment of abdominal aortic aneurysm.

### BACKGROUND OF THE INVENTION

1. Field of Invention

The invention relates to the field of medical devices, and more specifically to a prosthesis for the treatment of vascular disease, particularly abdominal aortic aneurysm.

2. Description of Related Art

Vascular disease is a leading cause of premature mortality in developed nations, often presenting as a vascular aneurysm. A vascular aneurysm is a localized dilation of a vessel wall, due to thinning or weakness of the wall structure, or separation between layers of the vessel wall. If untreated, the aneurysm may burst and hemorrhage uncontrollably. Aneurysms are particularly dangerous and prevalent in the aorta, because the aorta supplies blood to all other areas of the body, and because the aorta is subject to particularly high pressures and stresses accordingly. Rupture of an aortic aneurysm is the 15^{th} leading cause of death the United States, afflicting 5% of older men.

Aortic aneurysms are described by their position. They are either thoracic, generally between the aortic arch and the junction of the left and right renal arteries, or abdominal, between the junction of the renal arteries and the branch of the iliac arteries.

It is known to treat aortic aneurysms surgically where blood pressure control medication is unsuccessful at arresting growth of the aneurysm. Surgery often involves the insertion of a vascular stent graft to exclude the aneurysm and carry blood past the dilated portion of the vessel, relieving the pressure on the aneurysm. Designing a viable stent graft for the treatment of abdominal aortic aneurysm (AAA) is particularly challenging, in part because the graft must branch to follow the shape of the abdominal aorta to carry blood into the separate iliac arteries without obstruction.

Moreover, it would be advantageous to design a stent graft that is collapsible to facilitate percutaneous insertion by minimally invasive surgical techniques. Toward this end, certain stent grafts for the treatment of AAA are delivered in parts. One part of the stent graft forms a generally inverted Y shape, for insertion into the trunk of the abdominal aorta, and branching into the iliac arteries. A second part of the stent graft is deployed in the iliac artery and interfaces with the first part.

However, the stent graft must remain in the proper position after deployment and for years of continuous use. Accordingly it would be advantageous to design a multi-part stent graft that can be secured in position by minimally invasive surgical techniques. Further, it would be advantageous for a stent graft to be sizable in situ without compromising positional stability once deployed.

### BRIEF SUMMARY OF THE INVENTION

Provided according to the present invention is a prosthetic stent graft including a trunk with a bifurcated vascular graft supported by first one or more stent sections, preferably shape memory material and more preferably Nitinol. An inlet bifurcates into two flow channels in fluid communication with two outlets. The flow channels have a first taper angle, where the flow channel decreases in cross-sectional area along its length between the bifurcation point and an outlet of the flow channel.

A leg section of the stent graft includes a tubular vascular graft supported by second one or more stent sections, also preferably shape memory material and more preferably Nitinol. A second inlet of the leg section has a deployed diameter at least as large or larger than a deployed diameter of the outlets of the flow channels, and a third flow channel in fluid communication with an outlet. The leg section has its own taper angle, whereby the leg section flow channel decreases in cross-sectional area along its length between the inlet of the leg section and a location along the length of the leg section.

The first taper angle of the flow channels is preferably greater than or more preferably equal to the taper angle of the leg section. Preferably, the deployed diameter of the leg section inlet is at least as large or larger than the deployed diameter of the flow channels adjacent the bifurcation point of trunk section. Preferably, the deployed diameter of flow channel outlets are at least smaller than the deployed diameter of the leg section at an equal distance from the leg section inlet as the outlet is from bifurcation point.

Also provided according to the present invention is a method of in situ stent graft sizing for the treatment of abdominal aortic aneurysm. As part of the method, a bifurcated trunk section of a stent graft is deployed in the abdominal aorta of a patient. Further, a leg section of the stent graft is deployed with the inlet of the leg section within one of the bifurcated flow channels of the trunk section, the outlet of the leg section extending into the iliac artery. The inlet of the leg section has a deployed diameter at least as large or larger than the outlet of the bifurcated flow channel it is deployed within.

### BRIEF DESCRIPTION OF THE DRAWINGS

These and other features, benefits, and advantages of the present invention will be made apparent with reference to the following detailed description, appended claims, and accompanying figures, wherein like reference numerals refer to like structures across the several views, and wherein:

FIG. 1 illustrates a bifurcated trunk section of a stent graft according to an exemplary embodiment of the present invention;

FIG. 2 illustrates a leg section of the stent graft according to an exemplary embodiment of the present invention;

FIG. 3 illustrates a bifurcated trunk section and leg section of the exemplary embodiment superimposed over one another at their respected maximum deployed diameters, and at a preferred maximum axial overlap between the sections; and

FIG. 4 illustrates a bifurcated trunk section and leg section of the exemplary embodiment superimposed over one another at their respected maximum deployed diameters, and at a preferred minimum axial overlap between the sections.

### DETAILED DESCRIPTION OF THE INVENTION

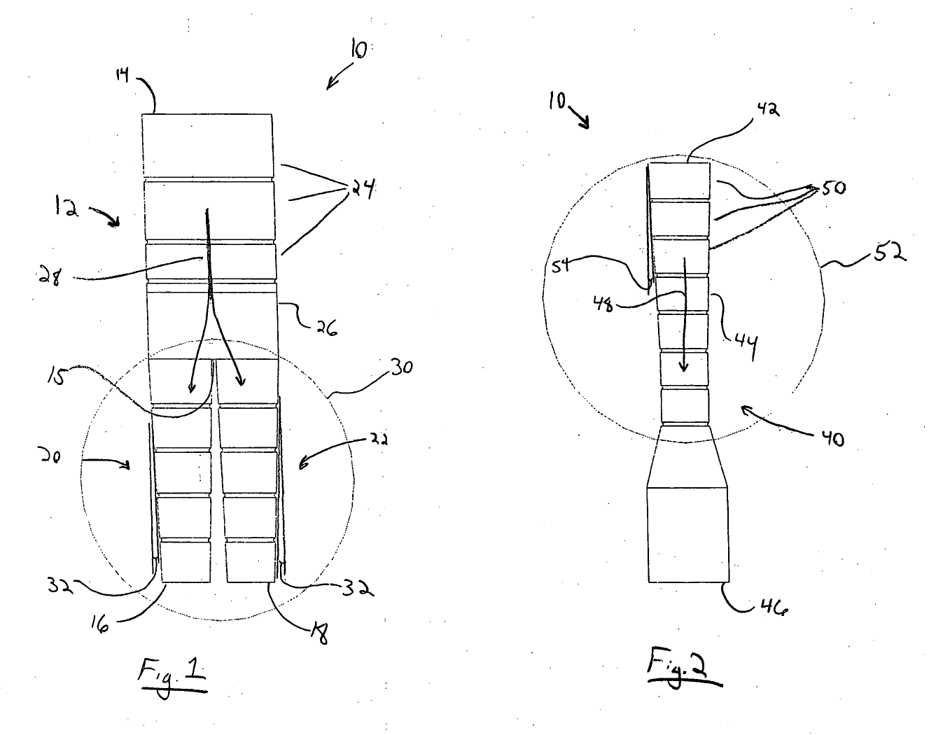

Referring now to FIGS. 1 and 2, illustrated are two components of stent graft, generally 10, according to the present invention. Trunk section 12 has a substantially circular inlet 14 at one end, and two substantially circular outlets 16, 18, at an opposite end. Inlet and outlet are derived from the intended direction of blood flow, indicated by arrow 28, through the trunk section 14 when positioned in the abdominal aorta and iliac arteries of a patient. The trunk section 12 bifurcates at point 15 between inlet 14 and outlets 16, 18 into two flow channels, 20, 22, respectively. Preferably, the trunk section 12 is comprised of plural stent segments 24 along its length, secured to and joined by a vascular graft 26 which surrounds the segments 24 and defines the flow through the trunk section 12. However, a unitary stent structure may be used as well, in addition to more or fewer segments 24 of varying length than shown in this exemplary embodiment. In a preferred embodiment, stent segments are approximately 1 cm in axial length.

Referring now to the bifurcated section highlighted by circle 30, according to the exemplary embodiment, the flow channels 20, 22 are each tapered, and reduce in diameter along their length between point 15 and outlets 16, 18.

Turning now to FIG. 2, stent graft 10 includes a leg section, generally 40. Leg section 40 has a generally circular inlet 42. Graft section 44 defines a flow path between inlet 42 and a generally circular outlet 46. Inlet and outlet are derived from the intended direction of blood flow, indicated by arrow 48, through the leg section 40 when positioned in the abdominal aorta and iliac arteries of a patient. Preferably, the leg section 40 is comprised of plural stent segments 50 along its length, secured to and joined by vascular graft 44 which surrounds the segments 50 and defines the flow through the leg section 40. However, a unitary stent structure may be used as well, in addition to more or fewer segments 50 of varying length than shown in this exemplary embodiment.

Referring now to the inlet section highlighted by circle 52, according to the exemplary embodiment, the leg section 40 is tapered, and reduces in diameter along its length at least between inlet 42 and some arbitrary point along its length. As shown in the exemplary embodiment, the leg portion 40 also expands in diameter from a minimum diameter along its length to a larger diameter at the generally circular outlet 46. However, this is merely optional. This expanded deployed diameter may be constrained by the vessel in which the outlet 46 of the leg portion 40 is deployed. Alternately, the taper may cease along its length, at which point the diameter may be arbitrarily set to suit the needs of the particular application, including continuing at a uniform diameter to the outlet 46. Alternately, the minimum diameter may itself occur at outlet 46.

Moreover, the taper angle 32, 54 is provided to improve fluid flow through the graft 10, by reducing flow turbulence. Reduced turbulence reduces the fluid pressures in the graft 10, thereby reducing stress on the graft 10, and particularly stent segments 24, 50 thereof. Accordingly the graft 10 is more resilient and enjoys longer service life.

Preferably, the stent segments 24, 50 forming the structure of stent graft 10 comprise a shape-memory material, more preferably Nitindl. Having been shape-set to a deployed diameter, the stent graft may be crimped to a smaller delivery diameter for percutaneous delivery by minimally invasive surgical techniques.

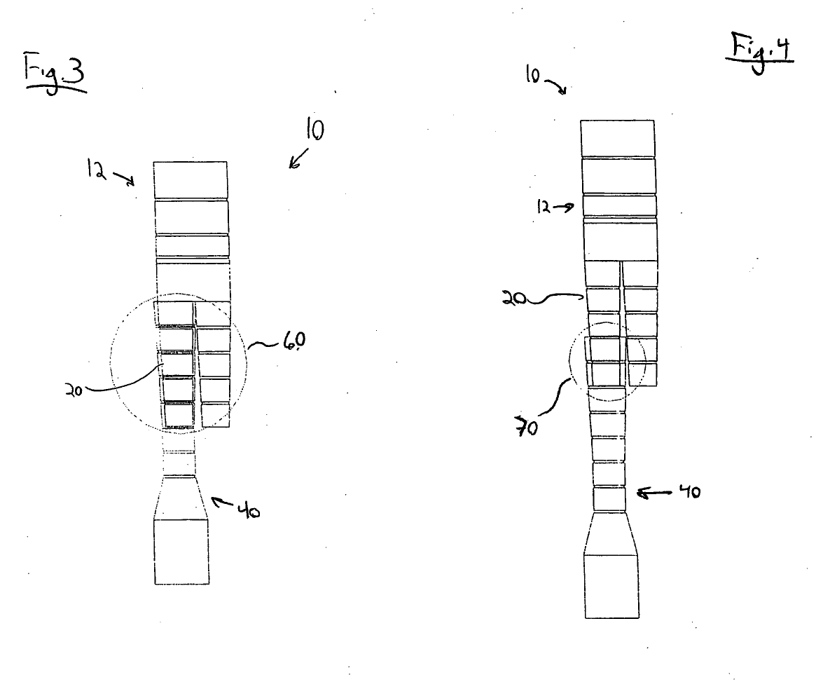

In use, the trunk section 12 of stent graft 10 would first be deployed in the abdominal aorta of the patient. Exposed to the heat of the patient's body temperature, and/or released from the constraint of a delivery apparatus, the stent graft 10 would expand to its deployed size, and fix against the walls of the aorta. Subsequently, one or two leg sections 40 would be deployed inside either or both of flow channels 20, 22. Preferably, the inlet 42 of leg section 40 would be deployed as close as practicable to the bifurcation point 15 of the trunk section 12. This affords the maximum overlap between the trunk section 12 and the leg section 40, which increases the pull-out force necessary to dislodge the leg section. Accordingly, the leg section 40 is more resistant to dislodgment. However, in practice, in situ sizing of the length of the stent graft 10 is often necessary, and the length of overlap between trunk section 12 and leg section 40 can be reduced as necessary (See, FIGS. 3-4).

The deployed diameter of the inlet 42 of leg section 40 is at least as large or larger than the deployed diameter of flow channels 20, 22 at their largest dimensions adjacent the bifurcation point 15 of trunk section 12. Additionally, the taper angle 32 of flow channels 20, 22, is at least approximately equal to or greater than the taper angle 54 of the tapered portion of leg section 40. Preferably, taper angle 32 and taper angle 54 are approximately equal. Therefore, presuming maximum overlap of the leg section 40 and flow channel 20, 22, the deployed diameter of the outlets 16, 18, is as least the same size or smaller than the corresponding deployed diameter of leg section 40 an equal distance from inlet 42 as outlet 16, 18 is from bifurcation point 15.

Accordingly, when a leg section 40 is inserted into a flow channel 20, 22, the deployed diameter of the leg section 40 would seek to exceed the inner deployed diameter of the flow channel 20, 22, but for containment of the leg section 40 by the flow channel 20, 22. The pull-out force necessary to dislodge the leg section from the flow channel increases with the amount by which the deployed diameter of the leg portion 40 exceeds the deployed diameter of the flow channel 20, 22, and also with the length of overlap between the leg section 40 and the flow channel 20, 22. Further, the pull-out force necessary to dislodge the leg section 40 increases with the taper angle 32.

Referring now to FIGS. 3 & 4, stent graft 10 is illustrated with leg section 40 superimposed over flow channel 20, both in fully deployed diameters, at a preferred maximum (FIG. 3) and preferred minimum (FIG. 4) amounts of axial overlap length between the two. It will be appreciated that more or overlap may be used, within the dimensional constraints of the leg section 40 and flow channel 20. In FIG. 3, it will be seen that the diameter differential between the leg section 40 and the flow channel 20 at any given point along their lengths is reduced by increasing amount of overlap, in conjunction with the taper angles 32, 54, highlighted by circle 60. Comparing now to FIG. 4, the axial overlap between leg section 40 and flow channel 20 is highlighted by circle 70. Although the lengths of the overlap is reduced as compared to FIG. 3, the differential in deployed diameter between leg section 40 and flow channel 20 is increased, in part due to the taper angles 32, 54. Any decrease in pull out force in the configuration of FIG. 4 because of the reduced overlap length is compensated for by the increase in pull out force by the increased differential in deployed diameter. Therefore, a surgeon can size the overall length of the stent graft 10 in situ without compromising pull-out force of the leg section 40.

The present invention has been described herein with reference to certain exemplary or preferred embodiments. These embodiments are offered as merely illustrative, not limiting, of the scope of the present invention. Certain alterations or modifications may be apparent to those skilled in the art in light of instant disclosure without departing from the spirit or scope of the present invention, which is defined solely with reference to the following appended claims.

1. A prosthetic stent graft comprising:
a trunk section having a bifurcated vascular graft having a first inlet and first and second flow channels in fluid communication with first and second outlets, the vascular graft supported by first one or more stent sections;
each of the first and second flow channels having a first taper angle, whereby the respective flow channel decreases in cross-sectional area along its length between a bifurcation point in the trunk section to an outlet of the flow channel;
a leg section having a tubular vascular graft having a second inlet with a deployed diameter at least as large or larger than a deployed diameter of the first or second outlet of the first or second flow channel, respectively, and a third flow channel in fluid communication with a third outlet, the vascular graft supported by second one or more stent sections; and
the leg section having a second taper angle, whereby the third flow channel decreases in cross-sectional area along its length between the second inlet and a location along the length of the leg section.

2. The prosthetic stent graft according to claim 1, wherein the first one or more stent sections or the second one or more stent sections comprise a shape memory material.

3. The prosthetic stent graft according to claim 2, wherein the shape memory material comprises Nitinol.

4. The prosthetic stent graft according to claim 1, wherein the first taper angle is greater than or equal to the second taper angle.

5. The prosthetic stent graft according to claim 1, wherein the first taper angle is substantially equal to the second taper angle.

6. The prosthetic stent graft according to claim 1, wherein the deployed diameter of the second inlet of the leg section is at least as large or larger than the deployed diameter of the first or second flow channel adjacent the bifurcation point of trunk section.

7. The prosthetic stent graft according to claim 1, wherein a deployed diameter of first or second outlet is as least the same as or smaller than the deployed diameter of the leg section at an equal distance from the second inlet as the first or second outlet is from bifurcation point.

8. The prosthetic stent graft according to claim 1, wherein the bifurcated vascular graft of the trunk section surrounds and is secured to the first one or more stent segments.

9. The prosthetic stent graft according to claim 1, wherein the tubular vascular graft of the leg section surrounds and is secured to the second one or more stent segments.

10. A method of in situ stent graft sizing for the treatment of abdominal aortic aneurysm, the method comprising:
(a) deploying a trunk section of a stent graft in the abdominal aorta of a patient, the trunk section having a bifurcated vascular graft having a first inlet and first and second flow channels in fluid communication with first and second outlets, the vascular graft supported by first one or more stent sections, each of the first and second flow channels having a first taper angle, whereby the respective flow channel decreases in cross-sectional area along its length between a bifurcation point in the trunk section to an outlet of the flow channel;
(b) providing a leg section having a tubular vascular graft having a second inlet with a deployed diameter at least as large or larger than a deployed diameter of the first or second outlet of the first or second flow channel, respectively, and a third flow channel in fluid communication with a third outlet, the vascular graft supported by second one or more stent sections, the leg section having a second taper angle, whereby the third flow channel decreases in cross-sectional area along its length between the inlet of the leg section and a location along the length of the leg section.
(c) deploying the leg section while the second inlet is within one of the first or second flow channels and the third outlet extending into the iliac artery.

11. The method according to claim 10, further comprising positioning the second inlet adjacent a bifurcation point of the trunk section.
